(19) United States
(12) Patent Application Publication
(10) Pub. No.: US 2024/0415443 A1
(43) Pub. Date: Dec. 19, 2024

(54) SYSTEMS AND METHODS FOR LOCATING SEIZURE ONSET ZONES FROM RS-FMRI IN PEDIATRIC PHARMACO-RESISTANT EPILEPSY USING DEEP LEARNING

(71) Applicants: Sandeep Gupta, Phoenix, AZ (US); Ayan Banerjee, Gilbert, AZ (US); Payal Kamboj, Tempe, AZ (US)

(72) Inventors: Sandeep Gupta, Phoenix, AZ (US); Ayan Banerjee, Gilbert, AZ (US); Payal Kamboj, Tempe, AZ (US)

(73) Assignee: Arizona Board of Regents on Behalf of Arizona State University, Tempe, AZ (US)

(21) Appl. No.: 18/741,240

(22) Filed: Jun. 12, 2024

Related U.S. Application Data
(60) Provisional application No. 63/508,244, filed on Jun. 14, 2023.

(51) Int. Cl. A61B 5/00; G06V 10/44; G06V 10/762; G06V 10/764; G06V 20/70; G16H 30/40

(52) U.S. Cl. CPC A61B 5/4094; G06V 10/44; G06V 10/762; G06V 10/764; G06V 20/70; G16H 30/40

(57) ABSTRACT

A computer-implemented system (“DeepXSOZ”) exploits synergy between deep-learning based spatial features and shallow-learning based expert knowledge encoding to identify Seizure Onset Zones based on spatiotemporal data captured during brain imaging. DeepXSOZ implements an independent component sorting technique that a) reduces expert sorting workload by 7-fold and b) enables the usage of rs-fMRI as a low-cost outpatient pre-surgical screening tool.

### CROSS-REFERENCE TO RELATED APPLICATIONS

This is a U.S. Non-Provisional Patent Application that claims benefit to U.S. Provisional Patent Application Ser. No. 63/508,244 filed 14 Jun. 2023, which is herein incorporated by reference in its entirety.

### FIELD

The present disclosure generally relates to medical imaging, and in particular, to a system and associated method for locating seizure onset zones using rs-fMRI and deep learning.

### BACKGROUND

According to the World Health Organization (WHO), around 50 million people worldwide suffer from epilepsy. Pharmaco-Resistant Epilepsy (PRE), which accounts for 30% of cases, occurs when a patient is not seizure free for at least 12 months through adequate trials of two tolerated and appropriately chosen anti-epileptic medications, and it immensely affects the patient's quality of life. The most effective treatment for PRE is surgical resection or ablation of the Seizure Onset Zone (SOZ), part of the brain where seizure originates. Recent studies have advocated for early diagnosis and surgery to avoid developmental complications which may cause sudden deaths. In fact, surgical intervention as early as three months is shown to have excellent postsurgical seizure frequency outcomes (Engel scores), with negligible risk of permanent morbidity. In children, risk of post-surgery developmental impairments necessitates precise localization of SOZ for PRE treatment success.

It is with these observations in mind, among others, that various aspects of the present disclosure were conceived and developed.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIG. 1 is a diagram showing a process for planning treatment for individuals with PRE using rs-fMRI imaging aided by a system (FIGS. 4A and 4B) for classifying Independent Components (ICs) of spatiotemporal imaging data outlined herein;

FIG. 2 shows a series of images showing noise ICs, RSN ICs and SOZ ICs;

FIG. 3 is a diagram showing a data pre-processing method for training a machine learning model of a system outlined herein to classify ICs;

FIGS. 4A and 4B are a pair of simplified diagrams showing an architecture of a system for classifying ICs that may be used to partially automate the treatment planning process of FIG. 1;

FIG. 5 is a graphical representation showing receiver operating characteristics and search space for identifying SOZs within spatiotemporal imaging data; and

FIG. 6 is a simplified diagram showing an exemplary computing system for implementation of the system of FIGS. 4A and 4B.

Corresponding reference characters indicate corresponding elements among the view of the drawings. The headings used in the figures do not limit the scope of the claims.

### DETAILED DESCRIPTION

Surgical disconnection of Seizure Onset Zones (SOZs) at an early age is the most effective treatment for Pharmaco-Resistant Epilepsy (PRE) in children. Presurgical localization of SOZs with intra-cranial EEG (iEEG) requires safe and effective depth electrode placement. Resting-state functional Magnetic Resonance Imaging (rs-fMRI) combined with signal decoupling using independent component (IC) analysis has shown promising SOZ localization capability that guides iEEG lead placement. However, SOZ ICs identification requires manual expert sorting of 100s of ICs per patient by the surgical team which limits the reproducibility and availability of rs-fMRI+iEEG based pre-surgical screening. Automated approaches for SOZ IC identification using rs-fMRI for pediatric PRE patients may use deep learning (DL) that encodes intricacies of brain networks from scarcely available pediatric data but ignores the plethora of expert knowledge, or shallow learning (SL) expert rule-based inference approaches that are incapable of encoding the full spectrum of spatial features. The present disclosure outlines a computer-implemented system (“DeepXSOZ”) that exploits the synergy between DL based spatial feature and SL based expert knowledge encoding to overcome performance drawbacks of these strategies applied in isolation. DeepXSOZ is a machine-expert collaborative IC sorting technique that: a) reduces expert sorting workload by 7-fold; and b) enables the usage of rs-fMRI as a low cost outpatient pre-surgical screening tool. Comparison of DeepXSOZ with state-of-art techniques using patient- and IC-level metrics on 52 children with PRE ranging from 3 months to 18 years old shows that DeepXSOZ achieves higher sensitivity of 89.79%, precision of 93.6% and accuracy of 84.6% for patient level metrics. Knowledge level ablation studies show a pathway towards maximizing patient outcomes while optimizing the machine-expert collaboration for various scenarios.

### SUMMARY

A system for identifying Independent Components featuring Seizure Onset Zones using spatiotemporal imaging data includes a processor in communication with a memory, the memory including instructions executable by the processor to: access spatiotemporal imaging data including independent component data of a plurality of independent components (ICs) of the spatiotemporal imaging data; determine, for an IC of the plurality of ICs and by a first machine learning model at the processor, a value of a first label of the IC, the first label being indicative of a noise class or a non-noise class associated with the IC determine, for the IC and by a second machine learning model at the processor, a value of a second label of the IC based on a plurality of features of the IC, the second label being indicative of a Resting State Network (RSN) activity class or a Seizure Onset Zone (SOZ) activity class associated with the IC; and assign an output label of the IC based on the value of the first label and the value of the second label associated with the IC, the output label indicating the noise class, the RSN activity class, or the SOZ activity class. The memory can further include instructions executable by the processor to generate a list of ICs having the output label indicating the SOZ activity class.

The first machine learning model can include a vision-based deep learning neural network model operable to evaluate noisiness of the IC based on the independent component data associated with the IC.

The memory can further include instructions executable by the processor to: extract, prior to determining the value of the second label, the plurality of features of the IC from the independent component data associated with the IC, including: information representing clusters present within the spatiotemporal imaging data and associated with the IC; information representing clusters present within the spatiotemporal imaging data associated with the IC that overlap within white matter areas and/or gray matter areas; information representing sparsity of an activelet basis representation of the IC; and information representing sparsity of a sine representation of the IC.

In some examples, the second machine learning model can be a linear-support vector machine model operable to evaluate an activity state of the IC based on the plurality of features associated with the IC. The second machine learning model may have been trained to determine the value of the second label based on features of the IC based on a training dataset, the training dataset including training IC data representing a plurality of training ICs having a plurality of training features, a first subset of the plurality of training ICs being associated with the RSN activity class and a second subset of the plurality of training ICs being associated with the SOZ activity class. The memory can include instructions executable by the processor to generate a training feature of a training IC associated with the SOZ activity class by application of a Synthetic Minority Oversampling Technique to spatiotemporal imaging data (e.g., to ensure class balance).

The memory can further include instructions executable by the processor to: assign a first output value indicating the noise class to the output label based on the value of the first label associated with the IC, the value of the first label associated with the IC indicating the noise class.

The memory can further include instructions executable by the processor to: assign a second output value indicating the SOZ activity class to the output label based on the value of the second label associated with the IC, the value of the second label associated with the IC indicating the SOZ activity class (where the value of the first label associated with the IC indicating the non-noise class).

The memory can further include instructions executable by the processor to: compare, for the IC, a posterior probability associated with the second label with respect to a threshold value, the first label of the IC indicating the noise class and the second label of the IC indicating the SOZ activity class; and assign, based on comparison between the posterior probability and the threshold value, the second output value indicating the SOZ activity class to the output label based on the value of the second label associated with the IC, the posterior probability exceeding the threshold value.

The memory can further include instructions executable by the processor to: assign a third output value indicating the RSN activity class to the output label based on the value of the second label associated with the IC, the value of the second label associated with the IC indicating the RSN activity class and the value of the first label indicating the non-noise class.

In a further aspect, a method for identifying Independent Components featuring Seizure Onset Zones using spatiotemporal imaging data includes: accessing, by a processor in communication with a memory, spatiotemporal imaging data including independent component data of a plurality of independent components (ICs) of the spatiotemporal imaging data; determining, for an IC of the plurality of ICs and by a first machine learning model at the processor, a value of a first label of the IC, the first label being indicative of a noise class or a non-noise class associated with the IC; determining, for the IC and by a second machine learning model at the processor, a value of a second label of the IC based on a plurality of features of the IC, the second label being indicative of a Resting State Network (RSN) activity class or a Seizure Onset Zone (SOZ) activity class associated with the IC; and assigning an output label of the IC based on the value of the first label and the value of the second label associated with the IC, the output label indicating the noise class, the RSN activity class, or the SOZ activity class.

Further, a non-transitory computer readable medium can include instructions encoded thereon that are executable by a processor to: access spatiotemporal imaging data including independent component data of a plurality of independent components (ICs) of the spatiotemporal imaging data; determine, for an IC of the plurality of ICs and by a first machine learning model at the processor, a value of a first label of the IC, the first label being indicative of a noise class or a non-noise class associated with the IC determine, for the IC and by a second machine learning model at the processor, a value of a second label of the IC based on a plurality of features of the IC, the second label being indicative of a Resting State Network (RSN) activity class or a Seizure Onset Zone (SOZ) activity class associated with the IC; and assign an output label of the IC based on the value of the first label and the value of the second label associated with the IC, the output label indicating the noise class, the RSN activity class, or the SOZ activity class.

### I. Introduction

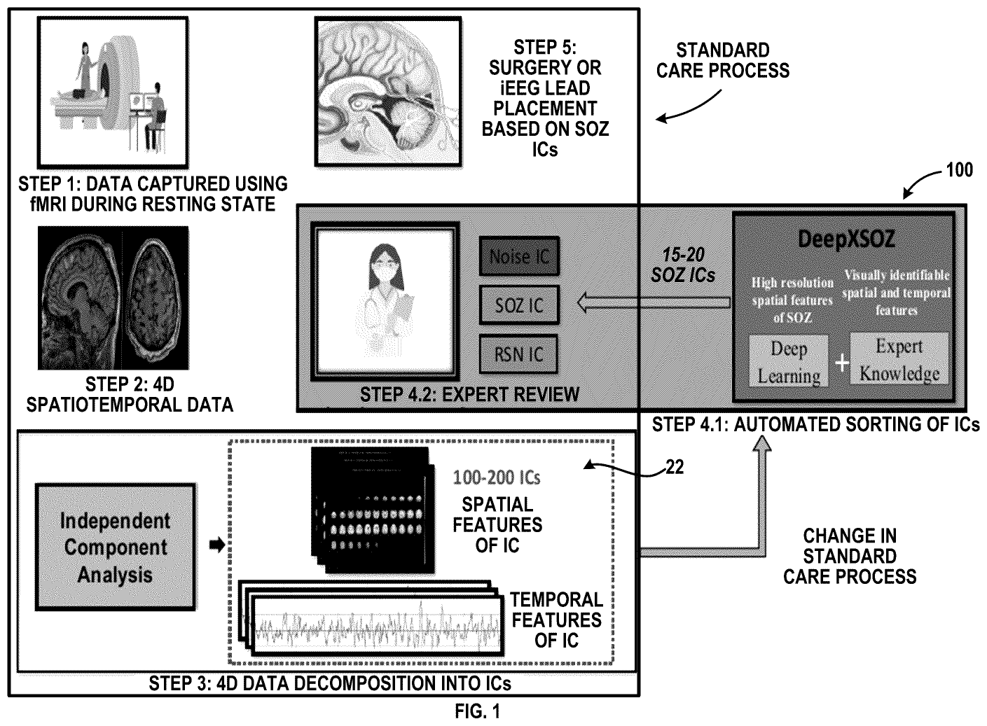

Seizures are spatio-temporal phenomena which originate from a SOZ with a typical onset duration between 4-8 seconds, and gradually propagate to other parts of the brain (also known as epilepsy networks, EN) with a speed of approximately 1 mm/s. As such, capturing SOZ using brain imaging is not only time sensitive but also requires high spatial resolution. Existing brain imaging techniques do not have the required spatial and temporal resolution for accurate SOZ identification. As a result, in standard pre-surgical evaluation (FIG. 1), SOZ is determined by multi-modal sensing such as the SISCOM method that combines ictal and inter-ictal single-photon emission computerized tomography (SPECT) scan, or a combination of Magnetoencephalography (MEG) for high temporal resolution and concordant functional Magnetic Resonance Imaging (fMRI) for high spatial resolution. The gold standard technique for localization of SOZ uses inter-cranial electroencephalography (iEEG), and requires implantation of depth electrodes. In children, such invasive methods come with risk of brain injury and long-term complications. Hence, placement of iEEG leads is guided by prior imaging-based analysis to avoid functional brain networks such as motor control or memory. fMRI is a non-invasive brain imaging technique that has high spatial resolution and has recently been shown to have 90% agreement with iEEG determined SOZ. Moreover, usage of fMRI in conjunction with iEEG to locate and surgically alter SOZ has shown significant improvement in surgical outcomes for children with PRE in terms of Engel scores without any developmental risks.

fMRI evaluates functional connectivity in terms of Blood Oxygen Level Dependent (BOLD) consumption activation (red colored clusters in step 2 of FIG. 1). It is a composite outcome of normal brain activity, seizure activity, and noise due to head movement or measurement artifacts. To decouple the brain activity from seizure activity, resting state is induced in PRE patients either through sedation or other means, resulting in the resting state fMRI (rs-fMRI). Independent component analysis (ICA) is a useful pre-processing technique for fMRI that generates mutually orthogonal spatio-temporal independent components (ICs) such that each IC encodes characteristics of either resting state brain activity, named as Resting State Network (RSN), or seizure onset, named as SOZ, or noise.

In standard pre-surgical evaluation (FIG. 1), the first step is to acquire rs-fMRI, which generates 4-D (3-D space and 1-D time) imaging data. The second step is pre-processing of the rs-fMRI for head motion artifact removal and ICA. “MELODIC” and “MCFLIRT” are popularly used software tools for this purpose. Based on the spatial and temporal resolution set at the measurement time, ICA of rs-fMRI may result in 100-200 ICs (right hand side of step 3 of FIG. 1). Although the ICs are mutually orthogonal, ICA cannot label the ICs as RSN, SOZ or noise. At this point, expert manual sorting is employed (Step 4).

This expert sorting of ICs not only requires careful observation, but it is a very time consuming and subjective process. This limits the reproducibility and availability of rs-fMRI based SOZ identification. DeepXSOZ (Step 4.1 in FIG. 1), fosters machine-expert collaboration to partially automate Step 4 in FIG. 1, and reduce manual sorting effort.

To reduce the need for expert IC sorting, automated machine learning (ML) based techniques for IC sorting have been explored recently (Tables I.A and I.B). Such techniques can be broadly classified into three classes: a) unsupervised techniques; b) shallow learning; and c) deep learning (DL). The most recent attempt, EPIK, is an unsupervised method, which has good accuracy and sensitivity, but has a significant number of false positives (FP) s, where non-SOZ brain parts are wrongly identified as SOZ. Shallow learning techniques suffer from significant false negatives (FN) s, where legitimate SOZ ICs are not identified. DL is a promising approach that has been explored in IC sorting for both healthy adults with n=2000 and on a limited dataset of children with PRE. While DL methods show >90% accuracy in identifying RSN, their sensitivity in identifying SOZ ICs was less than 20%. This is due to a major limiting factor for DL techniques that require balanced data across all the classes of interest.

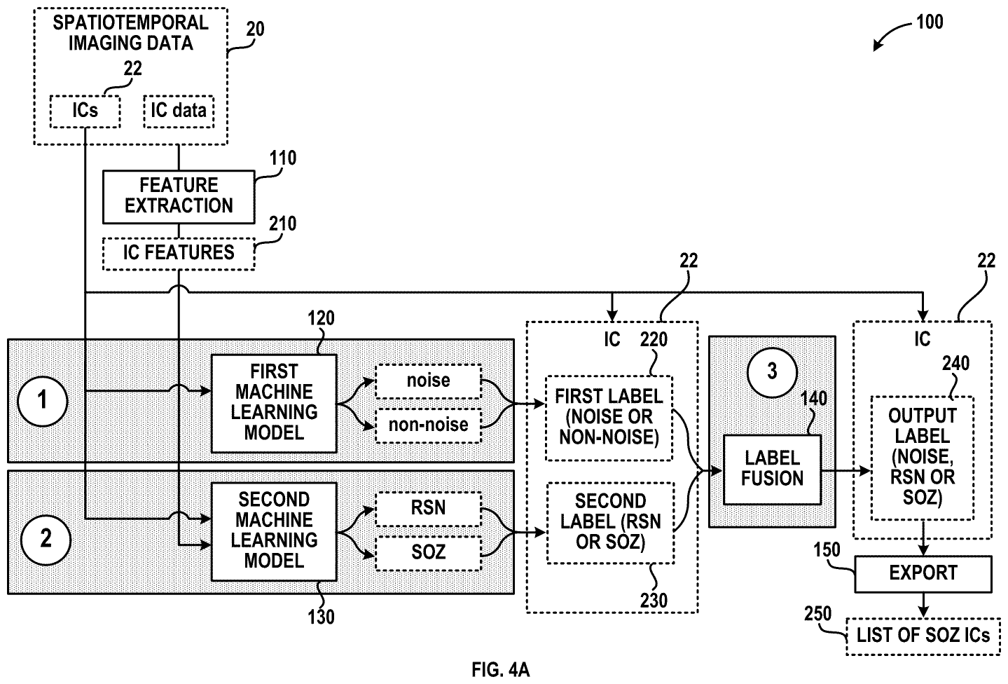

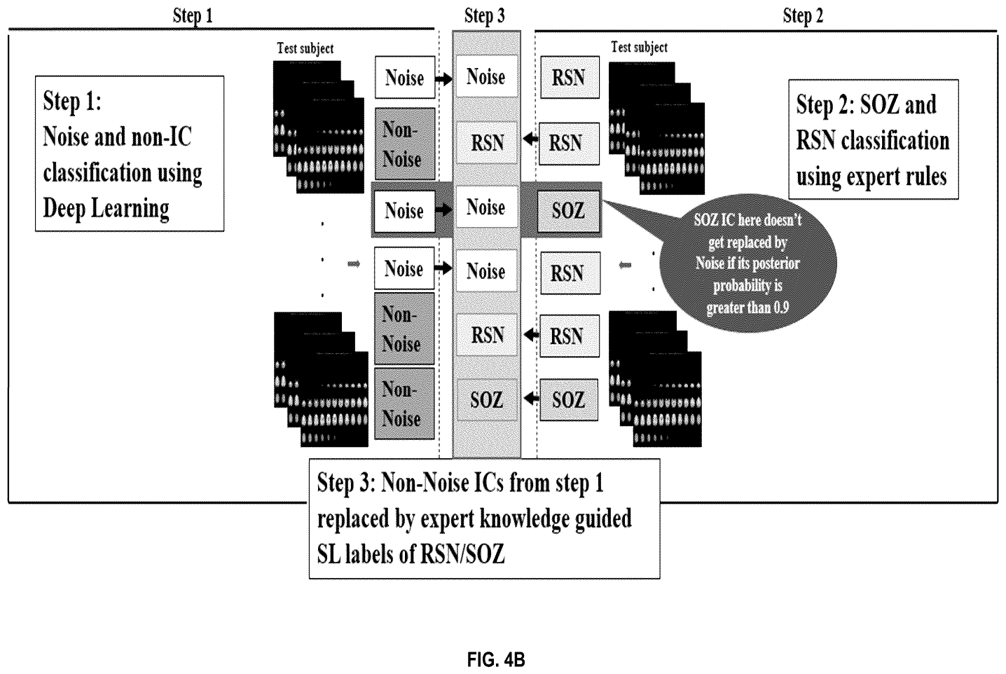

DL techniques have poor performance in applications with imbalanced data as shown in many different domains including fMRI image analysis. In medical imaging, the image capture protocols, as well as the psycho-physiological processes affecting the imaging outcome may change with several covariates such as age, sex, individual and family disease history, prior medical intervention and other co-occurring medical conditions. In such scenarios, DL performance may be negatively affected due to the lack of pathological data for a specific group of subjects especially the pediatric subgroup. To address such issues, expert knowledge can help to uniquely identify the characteristics of the imbalanced class (SOZ IC image in this case). DL techniques can be augmented with automated extraction of such knowledge with fast shallow learning techniques to achieve highly accurate localization of SOZ. DeepXSOZ (Step 3b box in FIG. 1, see also FIGS. 4A and 4B) utilizes the synergistic benefits of combining data driven DL techniques with expert knowledge guided shallow learning in the determination of SOZ ICs for children with PRE.

The present disclosure provides an evaluation of the performance of DeepXSOZ and other contemporary techniques with respect to two goals: a) statistical evaluation of DeepXSOZ in improving effectiveness of the pre-surgical screening of pediatric PRE population; and b) reducing manual IC sorting effort of the surgical team by optimizing machine-expert collaboration. To this effect, this disclosure uses patient level metrics, for the first evaluation goal, and IC level metrics, for the second goal.

### A. Summary of Contributions

FIG. 1 shows an rs-fMRI based pre-surgical screening workflow, that can be partially automated using a system 100 implementing “DeepXSOZ” outlined in FIGS. 4A and 4B. A primary aim of the system 100 outlined herein is to reduce the number of ICs that a surgical team would need to evaluate to determine iEEG lead placement or surgical resection/ablation procedure.

Contributions of the present disclosure include:
A system 100 applying a novel synergistic algorithm “DeepXSOZ” that combines DL classification results with expert knowledge on SOZ characteristics through shallow learning to achieve automated identification of SOZ localizing ICs which are relatively infrequent in the dataset. FIGS. 4A and 4B illustrate components and operation of the system 100.
DeepXSOZ partially automates Step 4 in the process shown in FIG. 1 and significantly reduces manual sorting effort from the neurosurgical team, achieving a 7-fold reduction in the number of ICs to be analyzed.
Comparison of DeepXSOZ with state of art shallow learning technique LS-SVM, CNN based DL technique and EPIK is presented on 52 children with PRE stratified across age, sex, and one-year post-operative Engel outcomes for rs-fMRI guided resection or ablation.
Evaluation using two types of metrics: a) patient level, for clinical efficacy analysis of pre-surgical screening; and b) IC level, for analysis of machine-expert collaboration.

### II. Related Technologies

Recent works broadly fall into two categories (Tables I.A and I.B): Epilepsy detection, which involves classification of patients as epileptic or non-epileptic based on EN identification, and SOZ localization, which is the main focus of this disclosure. Tables I.A and I.B provide a comparative analysis of recent works in terms of number of subjects, PRE subgroup proportion, age range, and the types of ICs that are identified. Works in this domain include varied evaluation metrics such as concordance with iEEG, agreement with expert identified SOZ, and consistency with physician assessment. In Table I.B, reported results column provides the evaluation metrics in the original manuscript for each work. Meta-data for each publication was recovered (whenever available or applicable) which enabled determination of patient level metrics and IC level metrics defined in Section IV-B.2 and IV-B.1, reported in the deduced results column.

For Tables I.A and I.B: not applicable (NA), accuracy (Acc), sensitivity (Sens), specificity (Spec), precision (Prec), not specified (NS), epilepsy networks (EN), epileptogenic zone (EZ), CD-cannot deduce, patient level metric (PLM, section IV-b.1), IC level metrics (ILM, section IV-b.2)), M in the “study” column indicates manual, A indicates automation.

TABLE IA
Review of fMRI based IC sorting

| Problem | Study | N | PRE | Age (years) | IC Class |
|---|---|---|---|---|---|
| Epilepsy | Nguyen (A) | 322 | 63 | Child (4-25) | NA |
| Detection | Lopes (A) | 15 | 0 | Adult (>18) | NA |
| | Bharath (A) | 132 | 0 | Adult (>18) | EN |
| SOZ | Boerwinkle (M) | 40 | 40 | Child (1.5-19.8) | EZ-SOZ |
| Localization | Gil (M) | 21 | 0 | Adult (>18) | SOZ |
| | Lee (M) | 29 | 29 | Adult (>18) | SOZ |
| | Hunyadi (A) | 18 | 18 | Adult (>18) | SOZ |
| | Nozais (A) | 2093 | 0 | Adult (>18) | RSN |
| | Luckett (A) | 2164 | 0 | Adult (>18) | SOZ |
| | Naresh (A) | 14 | 14 | Child (9-18) | EZ |
| | Zhang (A) | 10 | 10 | Adult (>18) | SOZ |
| | Banerjee (A) | 52 | 52 | Child (0.25-18) | RSN, SOZ |
| | Kamboj (A) | 52 | 52 | Child (0.25-18) | RSN, SOZ |

TABLE I.B
Review of fMRI based IC sorting (cont'd from Table I.A)

| Problem | Study | Reported Results | Deduced Results |
|---|---|---|---|
| Epilepsy | Nguyen (A) | Sens = 85%, | NA |
| | | Acc = 71%, | |
| | | Spec = 71% | |
| Detection | Lopes (A) | Acc = 87.5%, | NA |
| | Bharath (A) | Sens = 100%, | NA |
| | | Acc-97.5%, | |
| | | Spec = 94.4% | |
| SOZ Local- | Boerwinkle | Agreement with | PLM: Prec = 79%, |
| ization | (M) | iEEG derived | Sens = 93%, |
| | | SOZ = 90%, | Acc = 90% |
| | | Prec = 79%, | Spec: CD; ILM: CD |
| | | Sens = 93% | |
| | Gil (M) | NS | PLM: CD; ILM: CD |
| | Lee (M) | Concordance | PLM: Prec = 76%, |
| | | with iEEG | Sens = 76%, |
| | | derived SOZ = 72% | Acc = 72% |
| | | | Spec = 66%; ILM: CD |
| | Hunyadi (A) | PLM: Sens = 40%, | PLM, ILM reported |
| | | Acc = 51%, | herein |
| | | Spec = 77% | |
| | Nozais (A) | RSN Acc = 92%, | PLM: NA; ILM: NA |
| | Luckett (A) | Lateralization | PLM: NA; ILM: NA |
| | | of epilepsy foci | |
| | | Acc = 90% as | |
| | | compared to | |
| | | video EEG | |
| | Naresh (A) | PLM: Prec = 52%, | ILM: CD |
| | | Sens-40%, | |
| | | Acc = 88% | |
| | Zhang (A) | Consistency | PLM: Prec = 70%, |
| | | with physicians | Sens = 77%, |
| | | assessment | Acc = 41% |
| | | | Spec = 57%; ILM: CD |
| | Banerjee | PLM: Prec = 93%, | ILM reported herein |
| | (A) | Sens = 79%, | |
| | | Acc = 75% | |
| | Kamboj (A) | PLM: Prec = 93%, | ILM reported herein |
| | | Sens = 89%, | |
| | | Acc = 84% | |

Several manual SOZ identification techniques describe expert rules on SOZ specific spatio-temporal characteristics of BOLD signals captured by rs-fMRI. Boerwinkle et al. investigated the agreement between epileptogenic zone (EZ) by rs-fMRI and SOZ located by using iEEG data with prevalence-adjusted bias adjusted kappa (PABAK) on 40 patients and found the concordance to be 89%. Boerwinkle et al. revealed the weakness of previous techniques of using the most abnormal region of the brain to localize SOZ, however no work is reported on automation of expert sorted ICA-based SOZ classification. Gil manually studied 21 patients with extratemporal focal epilepsy to identify SOZ related ICs in fMRI data using the general linear model-derived EEG-fMRI time courses associated with epileptic activity. Lee et al. also manually investigated the functional connectivity changes in the ENs from rs-fMRI data using intrinsic connectivity contrast (ICC) to evaluate the non-invasive pre surgical diagnostic potential for SOZ localization. The agreement of fMRI-IC with intracranial EEG SOZ was 72.4%. The first automation attempts were from Hunyadi et al., who present a set of SOZ spatial and temporal features used to train a Least-Squares Support Vector Machine (LS-SVM). Evaluation on 18 pediatric PRE patients show high false negatives (FN).

DL was first explored by Nozais et al. to classify RSN ICs on non-PRE patients and reported an accuracy of 92%. However, they did not pursue SOZ identification. Luckett et al. used 2132 healthy control data for training of 3D CNN and tested it on temporal lobe epilepsy to detect the whole hemisphere of seizure onset. The training data was synthetically altered in randomly lateralized regions which helped in detection of biological SOZ's hemisphere. Note that ICs were not used here, so this work detected the whole brain hemisphere of seizure onset rather than the brain region pointing towards the SOZ. Their primary findings suggested the ICA guided by their technique has the potential to identify epilepsy-related ICs in patients with focal epilepsy. Naresh et al. explored deep graph neural networks using the T1 weighted images from rs-fMRI along with Diffusion MRI (dMRI) measurements. Study on 14 subjects showed a sensitivity of 40% and precision of 52% while an accuracy of 88%. This discrepancy in accuracy and precision is indicative of the problem of DL techniques with imbalanced data, where the unique characteristics of the rare class (SOZ in this case) is not captured due to lack of training data.

Zhang et al. proposed an ICA based automated method using unsupervised algorithm (UA) to localize the SOZ. SOZ ICs were screened based on peripheral noise IC removal, asymmetry and temporal features (excluding IC outside of frequency band 0.01-0.1 hz). Consistency with the resection surgery on 10 patients was reported. If consistency is assumed as true positive (TP), failure as FN and success in rejecting non-SOZ IC as true negative (TN) and failure to reject non-SOZ ICs as false positive (FP) then the results indicate significant FPs. The study by Banerjee et al. is the most recent study in the automation of SOZ localization. Banerjee et al. uses six expert features combined from Boerwinkle et al. and Hunyadi et al. (explained in detail in Section IV-A). This technique reports high accuracy but poor precision.

As such, the systems outlined herein implementing DeepXSOZ attempt to exploit “best of both worlds”, the capability of DL to extract spatial features and the capability of SL to encode expert knowledge.

### III. Methodology

### A. Data Collection

Retrospective analysis for this project was approved by the Phoenix Children Hospital (PCH), Institutional Review Board (IRB 20-358). Data collection included extraction of an rs-fMRI dataset of 52 children with PRE from the PCH clinical database.
1) Inclusion Criteria: Patients considered for inclusion in the dataset were determined to have PRE by a treating epileptologist and received surgery evaluation. Most of the patients had focal epilepsy, however, rapid generalization of epileptiform activity from an epileptogenic focus may appear to be generalized epilepsy when evaluated using surface EEG. Hence, generalized epilepsy was not an exclusion criterion.
2) Resting state fMRI collection method: The rs-fMRI data from 52 children with PRE, age 3 months-18 years old, were selected in descending alphabetical order from the PCH clinical database, who were under the care of a treating epileptologist at PCH (Table II). The diagnosis of PRE was according to the treating epileptologist's documented medical record notes. The children received rs-fMRI, video EEG, and anatomical MRI as part of standard clinical MRI SOZ localization for epilepsy surgery evaluation. For rs-fMRI, patients who were determined to require conscious sedation received a propofol infusion, as a part of standard care determined by the institution's policies. In the 52 children, 41 required conscious sedation. The dataset included only the patients who had less than 1 mm head motion in any direction during scanning. The MRI images were acquired using a 3T MRI unit, Ingenuity Philips Medical systems with a 32 channel head-coil. The rs-fMRI parameters were set at TR 2000 ms, TE 30 ms, matrix size 80×80, flip angle 80°, number of slices 46, slice thickness 3.4 mm with no gap, in-plane resolution 3×3 mm, interleaved acquisition, and number of total volumes 600, in two 10-min runs, with total time of 20 mins.

TABLE II
Patient Distribution

| | |
|---|---|
| Number of Subjects | 52 |
| Age ≤5 years | 20 |
| 5 < Age ≤13 years | 18 |
| 13 < Age ≤18 years | 14 |
| Male/Female | 23/29 |
| Prior surgery | 2 |
| Surgery post resting state fMRI | Ablation 15, resection 7 |
| Engel 1 score | Ablation 10, resection 6 |
| Engel 2 score | Ablation 5, resection 1 |
| Engel 3 score | Ablation 1, resection 0 |

3) rs-fMRI pre-processing: Oxford Center FMRIB (Functional MRI of the Brain) Software Library tool MELODIC was used to analyze the rs-fMRI and extract ICs. Pre-processing included deletion of the first 5 volumes to remove T1 saturation effects, passing through a high-pass filter at 100 seconds, slice time correction, spatial smoothing of 1-mm full-width at half maximum, and motion corrected by MCFLIRT, with nonbrain structures removed. Linear registration was performed between the individual functional scans and the patient's high-resolution anatomical scan which was further optimized using boundary-based registration. Individual rs-fMRI data sets then underwent ICA as previously reported.
4) Expert RS-fMRI evaluation methodology: The rs-SOZ was evaluated by the expert epilepsy surgery conference team and deemed to be consistent with the other acquired data (video EEG and anatomical MRI) with high enough evidence to surgically target the rs-SOZ. Further, the confirmation that the rs-SOZ was deemed true by the treatment team as evidenced by the Engel I and II scores 1 year post-operatively. The ICA results were viewed by two blinded reviewers (1 neurologist and 1 neurosurgeon) and sorted the ICs into 3 categories-noise, RSN, and rs-fMRI SOZ. In case of disagreement between the first two reviewers, the opinion of a third reviewer was used to make the final determination.

### B. DeepXSOZ Architecture and Process

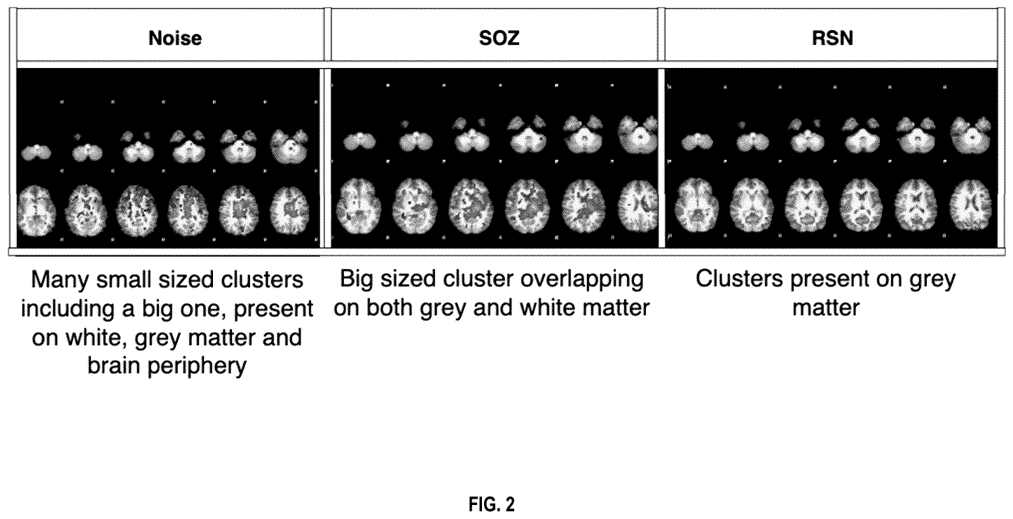

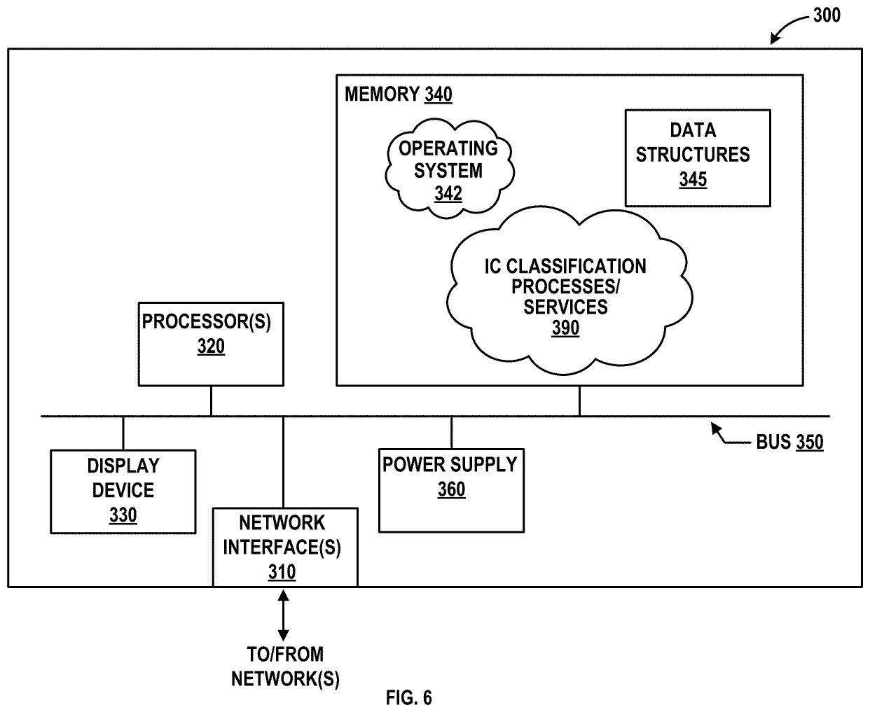

Referring to FIGS. 4A and 4B, a system 100 implementing DeepXSOZ can include a processor in communication with a memory (outlined further with respect to computing system 300 in FIG. 6). The processor can access spatiotemporal imaging data 20 including independent component data of a plurality of independent components (ICs) 22 of the spatiotemporal imaging data 20. FIGS. 1 and 2 show example ICs that correspond to decomposed spatiotemporal imaging data and may be classified into three classes: noise, RSN activity, or SOZ activity.

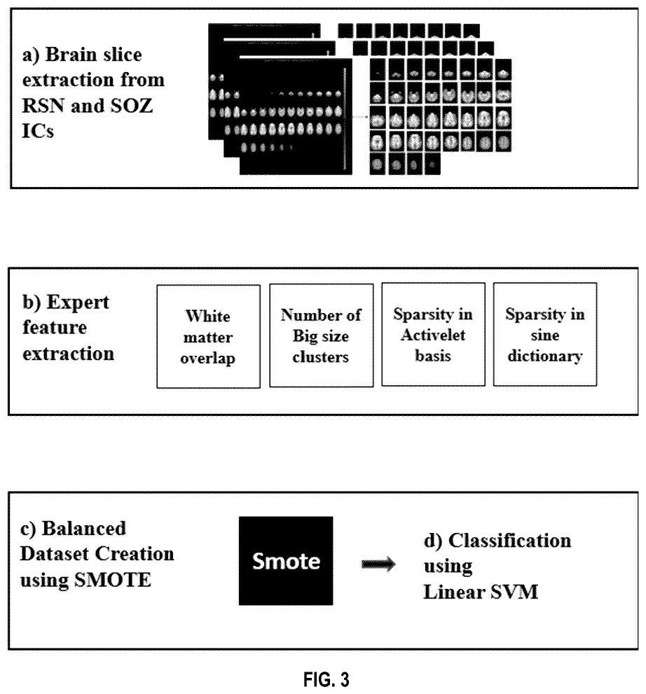

The system 100 can include a feature extraction module 110 that extracts a plurality of IC features 210 that can be used to classify ICs, particularly for classifying ICs under RSN or SOZ as in Step 2 discussed further herein. The plurality of IC features 210 can include, but are not limited to: information representing clusters present within the spatiotemporal imaging data and associated with the IC; information representing clusters present within the spatiotemporal imaging data associated with the IC that overlap within white matter areas and/or gray matter areas; information representing sparsity of an activelet basis representation of the IC; and information representing sparsity of a sine representation of the IC. Feature extraction may, in some examples, be performed with respect to a second machine learning model outlined herein associated with activity zone classification (Step 2). Further, feature extraction may also be applied during a training process for the second machine learning model, as outlined in FIG. 3.

### B1: Step 1: Noise Classification

The system 100 includes a first machine learning model 120 that determines, for an IC of the plurality of ICs, a value of a first label 220 of the IC, the first label 220 being indicative of a noise class or a non-noise class associated with the IC. In FIGS. 4A and 4B and in further discussion herein, this step is referred to as “Step 1” (or circle (1) in FIG. 4A), The first machine learning model 120 can include a vision-based deep learning neural network model operable to evaluate noisiness of the IC based on the independent component data associated with the IC.

### B2: Step 2: Activity Zone Classification.

The system 100 also includes a second machine learning model 130 that determines, for the IC, a value of a second label 230 of the IC based on the plurality of IC features 210 of the IC, the second label 230 being indicative of a Resting State Network (RSN) activity class or a Seizure Onset Zone (SOZ) activity class associated with the IC. In FIGS. 4A and 4B and in further discussion herein, this step is referred to as “Step 2”. The second machine learning model 130 can be a linear-support vector machine model operable to evaluate an activity state of the IC based on the plurality of features 210 associated with the IC.

Note that in some examples, the feature extraction module 110 can be integral with or otherwise associated with the second machine learning model 130, where an output of the feature extraction module 110 is used directly for the activity classification task in Step 2.

### B3: Step 3: Label Fusion

The system 100 can further include a label fusion module 140 that assigns an output label 240 of the IC based on the value of the first label 220 and the value of the second label 230 associated with the IC, the output label 240 indicating the noise class, the RSN activity class, or the SOZ activity class. In FIGS. 4A and 4B and in further discussion herein, this step is referred to as “Step 3”. FIG. 4B in particular shows the output classification strategy that fuses labels from the first machine learning model 120 and the second machine learning model 130.

For ICs having a value of the first label indicating the noise class, the label fusion module 140 can assign a first output value indicating the noise class to the output label 240 based on the value of the first label 220 associated with the IC.

For ICs having a value of the first label 220 indicating the non-noise class, the label fusion module 140 can use the values of the second label 230 to assign the value of the output label 240. In particular, for ICs having a value of the second label 230 indicating the SOZ activity class, the label fusion module 140 can assign a second output value indicating the SOZ activity class to the output label 240. For ICs having a value of the second label 230 indicating the RSN activity class, the label fusion module 140 can assign a third output value indicating the RSN activity class to the output label 240.

In some situations where the first machine learning model 120 classifies an IC as noise but the second machine learning model 130 classifies an IC as an SOZ with a posterior probability that meets or exceeds a threshold value (e.g., 90%), then the label fusion module 140 can assign, based on comparison between the posterior probability and the threshold value, the second output value indicating the SOZ activity class to the output label 240.

### B4: Output of DeepXSOZ

The system 100 can include an exporter module 150 that generates a list of ICs 250 having the output label 240 indicating the SOZ activity class, the RSN activity class, or the noise class. The list of ICs 250 can be displayed in a human-readable format at a display device (e.g., display device 330) along with their associated IC data for review by a practitioner. The list of ICs 250 may also be stored at a memory for further use in a downstream task. In some examples, a computing device can use information within the list of ICs 250 to highlight regions that correlate with SOZs within imaging data.

### B5: Training Process

A first training step includes training a Deep Learning (DL) network (e.g., the first machine learning model 120) to classify ICs into a noise class and a non-noise class (which encompasses RSN and SOZ ICs). The goal of the first training step is to specifically enable the DL network to learn to identify the Noise ICs. In practice, Step 1 of FIGS. 4A and 4B relies on the first training step to train the first machine learning model 120.

A second training step directed to the second machine learning model 130 further extracts the features of RSN and SOZ ICs on the basis of expert knowledge and also generates synthetic features of SOZ using Synthetic Minority Oversampling Technique (SMOTE) to create a balanced dataset. This is followed by training for these features using a linear-Support Vector Machine (SVM), which can be the second machine learning model 130. Note that noise ICs are omitted for training in the second training step. In practice, Step 2 of FIGS. 4A and 4B relies on the second training step to train the second machine learning model 130.

In practice, Step 3 of FIGS. 4A and 4B includes classifying each test subject's IC using the first machine learning model 120 and the second machine learning model 130. This results in two output lists, one from Step 1 and another from Step 2. Considering the output list from Step 2 as the base list, the IC label of the output list of Step 2 is changed to Noise (e.g., as belonging to the Noise class) if that IC is classified as a Noise IC by Step 1 (with the exception of ICs which have a posterior probability of greater than a threshold for SOZ as identified by Step 2). Other labels in the base list may remain the same. As a result, Step 3 outputs an an updated list of test subject's ICs classified under a Noise class, an RSN activity class and an SOZ activity class.

### 1) Step 1: Noise ICs Detection Using DL:

In one example implementation, a 2D Convolutional Neural Network (CNN) architecture was used as the first machine learning model 120. Note that other implementations of the first machine learning model 120 can use any suitable vision-based deep learning model (including CNN). The following hyperparameters of the CNN were tuned using a hyperband algorithm from Keras-tuner with the least validation loss objective:
Number of convolutional layers: [3; 4; 5]
Number of units/filters per convolutional layer: minimum=32, maximum=512, default=128.
Number of neurons in dense layer: minimum=192, maximum=1024, step=256.
Learning rate: [0.01; 0.001; 0.0001]
Dropout rate: [0.2; 0.33; 0.4; 0.5; 0.66].

In an example implementation, an image data generator by Keras was used to generate batches of noise and non-noise IC images, and provided real-time normalization of the images by rescaling them. A validation split of 0.1 was applied. Using the flow from directory method, the IC images were resized from 1006×709×3 to 270×400×3 for faster computation. Further, a ‘binary’ class mode and an ‘rgb’ ‘color_mode’ were used. ‘Binary cross-entropy’ was used as a loss function and ‘Adam’ was used as an optimizer. To avoid the overfitting problem, regularization methods including “dropout” and “early stopping” strategies were implemented. “ReLU”, being more computationally efficient, was used as an activation function for the input and hidden layers, and a “Sigmoid” activation function was used for the output layer. For CNN, weights were initialized using the “HeUniform” initializer. As images used within the dataset had dark backgrounds, and it was necessary to extract the sharp features as well as reduce the variance and computation complexity, a max pooling layer of 2×2 was implemented after every convolutional layer. The optimized hyperparameters' values given by the Keras-tuner were as follows: Number of convolutional layers: 3, number of 3×3 filters in convolutional layer 1, 2 and 3:64, 64 and 256 respectively, number of neurons in the dense fully connected layer: 704, learning rate: 0.0001 and dropout rate of 0.33.

Other deep learning techniques for noise and non-Noise ICs classification (Table III) were also implemented for development of the first machine learning model 120 to determine which had the most ideal performance:

Multilayer Perceptron (MLP): MLP was trained with the best hyperparameters using a Keras-tuner for the noise and non-noise ICs. This is similar to Nozais et al.

Transfer Learning: The VGG16 pre-trained model was used as a feature extractor, while a custom classifier model was implemented. Pre-trained weights of ‘imagenet’ in Keras were used, with addition of “flatten”, “dense” and “output” layers after the last layer in VGG16. The dense layer's number of neurons were tested for 5 different values: 192, 256, 512, 712 and 1024, out of which 512 gave the best accuracy. Fine-tuning the VGG model by freezing a few of its layers were not encouraging.

Problem Reduction: MELODIC software decomposes the 4D data of fMRI into spatial and temporal components. The images of the BOLD signal time courses and power spectrum from rs-fMRI were used to train the CNN model.

TABLE III
DL Techniques for Noise and Non-Noise ICs Classification

| Technique | Accuracy | Precision | Sensitivity | Specificity |
|---|---|---|---|---|
| CNN | 80.315% | 81.8% | 76.72% | 83.4% |
| MLP | 54.037% | 52.4% | 60.4% | 47.8% |
| Transfer Learning | 75.599% | 76.2% | 77.8% | 73.1% |
| Problem Reduction | 74.510% | 72.6% | 75.5% | 73.5% |

In an example embodiment, code for implementing the first machine learning model 120 that performs Step 1 was written in Python 3.7. All computations were run on Intel® Core™ i7-4790 CPU @ 3.60 GHz with 32 GB RAM and 64-bit operating system. Out of all techniques, CNN was the clear winner (also demonstrated in Table III) in eliminating the Noise ICs. For evaluation of noise and non-Noise ICs, True Positives (“TPs”) were defined as ICs that are classified as noise by both expert and the CNN, True Negatives (“TNs”) were defined as ICs that are classified as non-noise by both expert and the CNN, False Positives (“FPs”) were defined as ICs classified as non-noise by expert but noise by CNN, and False Negatives (“FNs”) were defined as ICs classified as noise by experts but non-noise by CNN.

### 2) Step 2: Feature Extraction Using Expert Knowledge:

FIG. 3 shows a general sequence for pre-processing data prior to application to the second machine learning model 130, which may take place during training of the second machine learning model 130. FIGS. 4A and 4B show operation of the second machine learning model 130 in the context of the system 100.

To extract features prior to classification at Step 2, the SOZ-specific expert rules explained by Hunyadi et al. and Boerwinkle et al. were used. The feature extraction module 110 can extract IC features 210 which include but are not limited to the following:
Number of clusters: An SOZ IC ideally has one cluster whereas an RSN IC includes multiple clusters.
White matter overlap: Big-sized cluster overlapping on the white matter (as well as gray matter) gives a good indication of the SOZ presence.
Sparsity in activelet basis: Activelets are a dictionary of wavelet basis functions which fit the BOLD signals. SOZ signal is made up of sparse transient events and thus has a sparse representation in the activelet basis.
Sparsity in sine dictionary: Time courses of RSN are characterized by low frequency (0.01-0.1 Hz) signals and thus show a sparse representation in this frequency band.

The IC features 210 extracted by the feature extraction module 110 can be applied as input to the second machine learning model 130 to assign the second label 230 to each IC. The feature extraction module 110 can be a component upstream from the second machine learning model 130 which uses these extracted features as data for training.

Step 2 considers whether an IC belongs to the RSN activity class or the SOZ activity class, ignoring the potential for noise (which is reconciled in Step 3, based on the output of Step 2). Step 2 can be further categorized in four parts.
Slice extraction: This part extracts the brain slice images from RSN and SOZ ICs so that expert guided features may be further extracted from these slices.
Expert features extraction: For each slice, a number of clusters is estimated using density-based spatial clustering of applications with noise (DBSCAN). Sobel filter-based edge detection technique was used to derive the contours for every slice where white matter demonstrates the brightest contour in the slice. Then, an overlap count of the big clusters on the white matter and/or gray matter is examined. ICs were also analyzed for activelet and sine dictionary sparsity in their time courses. For calculating the sparsity in activelet basis, the BOLD signal was divided into windows of length 256. From every window, four levels of activelet transformation coefficients using the ‘à trous’ algorithm with exponential-spline wavelets were extracted. The Gini Index metric was used for activelet coefficients and sine dictionary sparsity evaluation in the frequency band of 0.01 Hz to 0.1 Hz. As discussed, the feature extraction module 110 can be associated with the second machine learning model 130 and feature extraction may be performed as part of Step 2. Extracted features can be used as input to the second machine learning model 130 during training.
Balanced Dataset creation: Synthetic SOZ features are generated using SMOTE. Since the number of SOZ ICs available is limited, approximately 6 SOZ ICs per subject, SMOTE selects the real SOZ ICs samples in the feature space, and linearly interpolates features. SMOTE creates synthetic features for SOZ ICs based on just extracted expert knowledge-based features (e.g., as provided by feature extraction module 110. This is done to balance the dataset to feed it to the second machine learning model 130 for training.
Shallow learning for classification: The second machine learning model 130 (classifier) can be trained using expert driven features from RSN, noise and SOZ using SVM with linear kernel and Radial Basis Function (RBF) kernel, and it was found that the performance for the linear kernel was far better. k (52)-fold cross validation was used to quantify the classifier's accuracy. Every patient got tested using the k−1 training datasets. Similarly, k-fold cross validation was performed for every patient using CNN to get the noise and non-noise ICs in step 1. Classifier code for an example implementation of the second machine learning model 130 that performs aspects of Step 2 was written in MATLAB R2021b on the same computing device as the first machine learning model 120 that performs aspects of as Step 1.

### 3) Step 3: Combining the Outputs of Step 1 and Step 2:

In Step 3 (FIG. 4B), the test subject's ICs have previously been applied to the first machine learning model 120 of Step 1, which labels the ICs as belonging to the noise class and the non-noise class. The same test subject's ICs have also previously been applied to the second machine learning model of Step 2, which labels the ICs as belonging to the SOZ activity class or the RSN activity class. An output listing of ICs from Step 2 can be considered to be a “base list”, however, recall that the output list of Step 2 does not consider the noise class. As such, Step 3 includes re-labeling ICs in the base list that were classified under the noise class in step 1.

However, if there is an IC in the base list that was classified within Step 2 under the SOZ activity class with a posterior probability value higher than 0.9, then that IC remains within the SOZ activity class even if the trained machine of Step 1 had previously labeled the IC as belonging to the “noise” class. This action reduces false negatives.

### IV. Experiments and Results

This section of the disclosure provides an evaluation of the systems outlined herein. The evaluation has four goals: a) evaluate efficacy of Deep-XSOZ, its variation across age and sex and compare with state-of-the-art techniques; b) evaluate correlation of DeepXSOZ performance with surgical outcomes; c) knowledge ablation studies, to show relative importance of spatial and temporal expert knowledge on the DeepXSOZ performance; and d) evaluate the variation of manual IC sorting effort for different levels of reliance on the automation in DeepXSOZ.

### A. Comparative Techniques

Based on the related work in Tables I.A and I.B, the following state-of-art comparative techniques were selected for comparison with DeepXSOZ.
1) CNN Architecture: The CNN-based DL technique is similar to the technique applied in Step 1 (Section III-B.1) of DeepXSOZ, except that the output layer has three classes instead of two (e.g., which directly classifies an IC into a noise class, an SOZ activity class, or an RSN activity class without the multi-step evaluation process in DeepXSOZ). A cost-sensitive learning technique was applied for the CNN to give equal importance to all the classes on gradient updates.
2) LS-SVM based SOZ classification: The technique proposed in Hunyadi et al. was replicated for comparison to DeepXSOZ. For this technique, rs-fMRI IC's spatial and temporal signal features were extracted. To perform an unbiased comparison with DeepXSOZ, SMOTE was also applied to generate ICs with SOZ features and balance the three classes. The features extracted from the rs-fMRI IC spatial and temporal signals of Noise, RSN and SOZ were then used to train a LS-SVM as described in Hunyadi et al.
3) EPIK: Unsupervised Technique: EPIK (Banerjee et al.) uses six expert rules for an IC to be classified as noise, combined from Boerwinkle and Hunyadi's works (Tables I.A and I.B).

### B. Evaluation Metrics

Two classes of metrics are considered as observed in recent literature (Tables I.A and I.B):
1) Patient level metrics (PLM): These metrics are patient average. A “patient” is considered TP if automated technique identified at least one SOZ IC that was also manually identified as SOZ IC for that patient. A FP denotes that automated technique identified at least one SOZ IC, whereas no SOZ was identified manually for the patient. A FN denotes that automated technique could not find any SOZ IC, even though SOZ ICs were identified manually. A TN denotes that both automated technique and expert manual identification found no SOZ ICs. These metrics are commonly used in most recent works including Hunyadi et al., Zhang et al., and Lee et al., In case of focal epilepsy with only one SOZ, these metrics suffice. However, in case of multi-focal epilepsy such metrics inflate the success of the technique and may not indicate the multitude of SOZs. Accuracy, precision, and sensitivity are computed using the standard formula.
2) IC level metrics (ILM): These metrics are intended to evaluate the effort of the surgical team in identifying the SOZ. In specific, three metrics are considered:
i) Number of machine marked SOZ ICs: This is the total number of ICs marked as potential SOZ by any automated technique. This number is directly proportional to the manual sorting effort of the surgical team.
ii) SOZ incidence: Expressed as a percentage, this is the total number of SOZ ICs among the machine marked SOZ ICs.

SOZ Sensitivity: This metric measures the automated technique's capability in correctly identifying the SOZ ICs of a patient by measuring the ratio of total number of machine identified SOZ over total number of manually identified SOZs.

### C. Statistical Methods

Statistical methods are utilized to derive the significance of: a) the effect of age and sex on the ILM evaluations; and b) the difference in PLM among different algorithms. For the first aim, a mixed effects model is utilized where the age and sex along with their combined effect as predictors and random effect on the patient. For the second aim, a one sided t-test is utilized to evaluate statistical significance of the difference between DeepXSOZ and other comparative techniques. The 95% confidence p values are provided in Tables IV.A-IV.D.

TABLE IV.A
SOZ Identification Performance Metrics (where EoK denotes effect
of merging expert knowledge in DeepXSOZ)

| | | Age 0-5, | Age 5-13, | Age 13-18, |
|---|---|---|---|---|
| PLM | Method | N = 20 (EoK) | N = 18 (EoK) | N = 23 (EoK) |
| SOZ | DeepXSOZ | 80.0% | 88.8% | 85.7% |
| Accuracy | | (+30%) | (+50%) | (+42%) |
| | CNN | 50.0% | 38.8% | 42.8% |
| | EPIK | 90.0% | 72.2% | 64.2% |
| | LS-SVM | 31.5% | 61.1% | 71.4% |
| SOZ | DeepXSOZ | 94.1% | 100.0% | 85.7% |
| Precision | | (+3%) | (0%) | (+10%) |
| | CNN | 90.9% | 100% | 75.0% |
| | EPIK | 94.7% | 100% | 75.0% |
| | LS-SVM | 85.7% | 100% | 83.3% |
| SOZ | DeepXSOZ | 84.2% | 88.8% | 100% |
| Sensitivity | | (+31%) | (+50%) | (+50%) |
| | CNN | 52.6% | 38.8% | 50.0% |
| | EPIK | 94.7% | 72.2% | 75.0% |
| | LS-SVM | 33.3% | 61.1% | 83.3% |

TABLE IV.B
SOZ Identification Performance Metrics (where EoK
denotes effect of merging expert knowledge in DeepXSOZ)

| | | Male, | Female | Overall | |
|---|---|---|---|---|---|
| | | N = 23 | N = 29 | Results | DeepXSOZ |
| PLM | Method | (EoK) | (EoK) | (EoK) | compare p |
| SOZ | DeepXSOZ | 91.3% | 79.3% | 84.6% | NA |
| Accuracy | | (+52%) | (+37%) | (+38%) | |
| | CNN | 39.1% | 41.9% | 46.1% | p ≈ 0 |
| | EPIK | 78.2% | 72.4% | 75.0% | p = 0.08 |
| | LS-SVM | 60.8% | 44.8% | 50.0% | p ≈ 0 |
| SOZ | DeepXSOZ | 95.4% | 95.8% | 93.6% | NA |
| Precision | | (+5%) | (+9%) | (+5%) | |
| | CNN | 90.0% | 86.6% | 88.8% | p = 0.02 |
| | EPIK | 94.7% | 91.3% | 92.8% | p = 0.2 |
| | LS-SVM | 93.3% | 86.6% | 89.6% | p = 0.03 |
| SOZ | DeepXSOZ | 95.4% | 82.1% | 89.7% | NA |
| Sensitivity | | (+54%) | (+37%) | (+41%) | |
| | CNN | 40.9% | 44.8% | 48.9% | p ≈ 0 |
| | EPIK | 81.8% | 77.7% | 79.5% | p = 0.1 |
| | LS-SVM | 63.6% | 48.1% | 53.6% | p ≈ 0.0008 |

TABLE IV.C
SOZ Identification Performance Metrics

| | | Age 0-5, | Age 5-13, | Age 13-18, | Age |
|---|---|---|---|---|---|
| ILM | Method | N = 20 (SD) | N = 18 (SD) | N = 14 (SD) | (p) |
| Machine | DeepXSOZ | 21 (11) | 17 (6) | 16 (4) | 0.7 |
| Marked | CNN | 12 (30) | 9 (18) | 9 (17) | 0.8 |
| SOZs (SD) | EPIK | 39 (10) | 45 (9) | 48 (10) | 0.5 |
| | LS-SVM | 6 (6) | 4 (6) | 6 (2) | 0.7 |
| SOZ | DeepXSOZ | 11.1% (11) | 17.2% (11) | 13.5% (8) | 0.4 |
| Incidence | CNN | 23.9% (33) | 9.8% (18) | 14.5% (28) | 0.56 |
| (SD) | EPIK | 5.8% (3) | 5% (4) | 3.4% (3) | 0.42 |
| | LS-SVM | 8.4% (14) | 19.1% (18) | 13.6% (16) | 0.63 |
| SOZ IC | DeepXSOZ | 29% (27) | 39.81% | 51% (30) | 0.4 |
| Sensitivity | | | (23) | | |
| (SD) | CNN | 25.4% (42) | 21.3% (36) | 22% (31) | 0.8 |
| | EPIK | 41.6% (27) | 31.5% (27) | 32.6% (27) | 0.4 |
| | LS-SVM | 9.6% (17) | 21.1% (27) | 32.4% (30) | 0.8 |

TABLE IV.D
SOZ Identification Performance Metrics

| | | Male, | Female, | | Overall | |
|---|---|---|---|---|---|---|
| | | N = 23 | N = 18 | | Results | DeepXSOZ |
| ILM | Method | (SD) | (SD) | Sex (p) | (SD) | compare p |
| Machine | DeepXSOZ | 16 (5) | 20 (9) | 0.1 | 18 (8) | NA |
| Marked | CNN | 6 (13) | 14 (29) | 0.5 | 10 (23) | 0.02 |
| SOZs | EPIK | 44 (10) | 43 (10) | 0.2 | 43 (10) | ≈0 |
| (SD) | LS-SVM | 6 (4) | 6 (5) | 0.7 | 6 (4) | ≈0 |
| SOZ | DeepXSOZ | 17.3% (11) | 11.3% (11) | 0.07 | 13.8% (10) | NA |
| Incidence | CNN | 13.5% (24) | 12.4% (23) | 0.6 | 13.3% (24) | 0.17 |
| (SD) | EPIK | 5.4% (4) | 4.2% (4) | 0.5 | 4.7% (4) | ≈0 |
| | LS-SVM | 18.1% (18) | 16.4% (16) | 0.33 | 13.1% (18) | 0.96 |
| SOZ IC | DeepXSOZ | 41.3% (24) | 37.7% (29) | 0.1 | 39.3% (27) | NA |
| Sensitivity | CNN | 14.2% (25) | 20.5% (36) | 0.9 | 23.1% (37) | 0.37 |
| (SD) | EPIK | 34.5% (28) | 31.4% (26) | 0.1 | 33.5% (27) | ≈0 |
| | LS-SVM | 21.3% (26) | 17.9% (25) | 0.4 | 18.8% (26) | 0.009 |

### D. Results

The results in Tables IV.A-IV.D show that DeepXSOZ outperforms the LS-SVM, CNN and EPIK in all performance metrics. The table shows PLM and ILM across age and sex. For PLM, the effect of merging expert knowledge (EoK) with DL in DeepXSOZ is shown by the difference between DeepXSOZ and CNN. The last column provides the statistical significance of the difference between DeepXSOZ and other methods. At PLM, the significantly higher sensitivity of DeepXSOZ shows that there are very few FNs as compared to LS-SVM, CNN and EPIK which means DeepXSOZ is able to detect the correct SOZ ICs for pediatric PRE patients. At IC level DeepXSOZ on an average outputs 18 machine marked SOZs which are notably fewer than prior techniques. This suggests that DeepXSOZ may save manual sorting effort for the surgical team. A higher SOZ incidence indicates that the surgical team will have greater confidence in sorting through the low number of ICs. The ILM of SOZ sensitivity also conveys the improved capability of DeepXSOZ in classifying the correct SOZ ICs as compared to the other techniques. Tables IV.A-IV.D also show the DeepXSOZ's comparison with prior techniques for SOZ identification with respect to age and sex of the patients. At PLM, the current technique maintains statistically stable and higher accuracy, precision and sensitivity across all the age groups and sex distribution. Whereas LS-SVM and CNN show significant age and sex-based variance, which is indicative of the difference in data size rather than the main predictor. EPIK, which was not only statistically stable but the second-best performer after DeepXSOZ at patient level, failed at IC level with only 4.78% SOZ IC detection capability out of 43 machine marked SOZs. The p values listed in Tables IV.A-IV.D demonstrate the statistically significant difference between DeepXSOZ and either LS-SVM or CNN for PLM. However, statistically, there is insignificant difference between DeepXSOZ and EPIK. For ILM it is observed that none of the techniques have any significant effect of age or sex. Here, DeepXSOZ has statistically significant advantage over EPIK.
1) Surgical team effort reduction: DeepXSOZ reduces the time-commitment by the neurosurgeons in hand sorting the medical images by nearly 80%. Out of 100-140 ICs, Deep-XSOZ outputs approximately 18 potential SOZ ICs which are guaranteed to include all SOZs in them required for the pre-surgical evaluation. Effective and accurate removal of noise drastically reduces the time-consuming process of the neurosurgeons for going through all the ICs of the patient to locate the SOZ regions. Out of 49 patients, DeepXSOZ was able to correctly identify the SOZ ICs for 44 patients, giving an accuracy of 84.61%. The only five patients DeepXSOZ missed had on an average of only two SOZ ICs per patient, making it equally challenging for the neurosurgeons to locate them. The remaining three patients out of 52 total did not have any SOZ ICs present in the dataset provided by the expert.
2) Performance on subjects undergoing surgery: 24 subjects in total underwent surgery out of which, 16 subjects became seizure free (Engel I) after respective surgery of the identified SOZ using rs-fMRI ICs and 7 subjects displayed reduced post operative seizure frequency (Engel II). Tables V.A and V.B show the DeepXSOZ, LS-SVM and CNN performance for the SOZ identification on the 24 subjects that underwent surgery. For the patients undergoing ablation surgery, DeepXSOZ showed a higher sensitivity of 93.33%. The results are very encouraging given that ablation is minimally invasive and thus highly preferred over resection. The performance of DeepXSOZ is consistent on the patients undergoing resection with sensitivity of 85.71% whereas other techniques showed poor performance on the provided metrics due to the presence of a high number of FNs. Moreover, DeepXSOZ shows 93% agreement with expert sorting for patients with Engel 1 outcome. This increases confidence in usage of DeepXSOZ in pre-surgical screening.

TABLE V.A
Performance comparison of methods across surgical procedures

| | Ablation Procedures | | Resection Procedures | |
|---|---|---|---|---|
| | (N = 15) | | (N = 7) | |
| | | SOZ Incidence | | SOZ Incidence |
| Approach | Sensitivity | (SD) | Sensitivity | (SD) |
| DeepXSOZ | 93.33% | 13.7% (9) | 85.71% | 15.3% (10) |
| LS-SVM | 66.66% | 16.6% (19) | 57.14% | 15.4% (16) |
| CNN | 33.33% | 11.7% (20) | 42.85% | 9.2% (11) |
| EPIK | 73.33% | 7.9% (6) | 71.4% | 5.9% (5) |

TABLE V.B
Performance comparison of methods across Engel outcomes

| | Engel 1 Outcomes | | Engel 2 Outcomes | |
|---|---|---|---|---|
| | (N = 16) | | (N = 5) | |
| | | SOZ Incidence | | SOZ Incidence |
| Approach | Sensitivity | (SD) | Sensitivity | (SD) |
| DeepXSOZ | 93.75% | 15.6% (9) | 100% | 14% (3) |
| LS-SVM | 56.25% | 16.3% (19) | 80% | 15.3% (11) |
| CNN | 43.75% | 10.5% (15) | 60% | 19.3% (25) |
| EPIK | 75% | 6.9% (5) | 100% | 5.1% (3) |

### E. DeepXSOZ Knowledge Ablation Studies

The effect of removing knowledge components from DeepXSOZ on its performance is evaluated with respect to the PLM and ILM. The training data size of DeepXSOZ is also varied from 20% to 80% to derive receiver operating characteristics (ROC) curve, which gives insight on the trade-off between computational complexity, need for training data, level of reliance on automation, and SOZ identification performance.

DeepXSOZ without temporal features: The BOLD signal temporal features are removed one by one from the shallow learning component of DeepXSOZ. Three unique configurations are considered: a) DeepXSOZ without activelet domain sparsity; b) DeepXSOZ without sine domain sparsity; and c) Deep-XSOZ without any temporal features. Table VI shows that there is a statistically insignificant effect on PLM, which indicates that removing temporal features have little effect on the presurgical evaluation of patients with DeepXSOZ. Removing temporal features marginally reduces the SOZ incidence and sensitivity and increases the machine marked SOZ resulting in increased (albeit minimal) workload for the surgical team.

DeepXSOZ without spatial features: The spatial features are removed from the shallow learning component one by one to create three unique configurations: a) DeepXSOZ without number of clusters; b) DeepXSOZ without white matter overlap; and c) DeepXSOZ without spatial features. At PLM, slight improvement was observed in all three metrics for both, DeepXSOZ without white matter and spatial features. However, these increased metrics were the result of huge machine marked ICs presence at ILM, decreased SOZ Incidence and increased sensitivity leading to increased effort by the surgical team. DeepXSOZ without number of clusters did show minimal decrease in machine marked SOZ ICs but this comes at the cost of reduced SOZ IC sensitivity and PLMs.

TABLE VI
Knowledge Ablation Study: Machine marked SOZ (MM SOZ), SOZ
Incidence (SOZ Inc), SOZ IC Sensitivity (SOZ Sens)

| PLM | Accuracy | Precision | Sensitivity |
|---|---|---|---|
| DeepXSOZ without temporal features | 84.6% | 93.6% | 89.7% |
| DeepXSOZ without activelet sparsity | 84.6% | 93.6% | 89.7% |
| DeepXSOZ without sine sparsity | 84.6% | 93.6% | 89.7% |
| DeepXSOZ without spatial features | 86.5% | 93.7% | 91.8% |
| DeepXSOZ without number of clusters | 75% | 92.8% | 79.5% |
| DeepXSOZ without white matter overlap | 86.5% | 93.7% | 91.8% |
| ILM | MM SOZ | SOZ Inc | SOZ Sens |
| DeepXSOZ without temporal features | 19 | 12.6% | 38.8% |
| DeepXSOZ without activelet sparsity | 19 | 13.4% | 39.2% |
| DeepXSOZ without sine sparsity | 18 | 13.6% | 37.8% |
| DeepXSOZ without spatial features | 51 | 6.2% | 53.6% |
| DeepXSOZ without number of clusters | 16 | 13.8% | 34.8% |
| DeepXSOZ without white matter overlap | 51 | 6.2% | 53.6% |

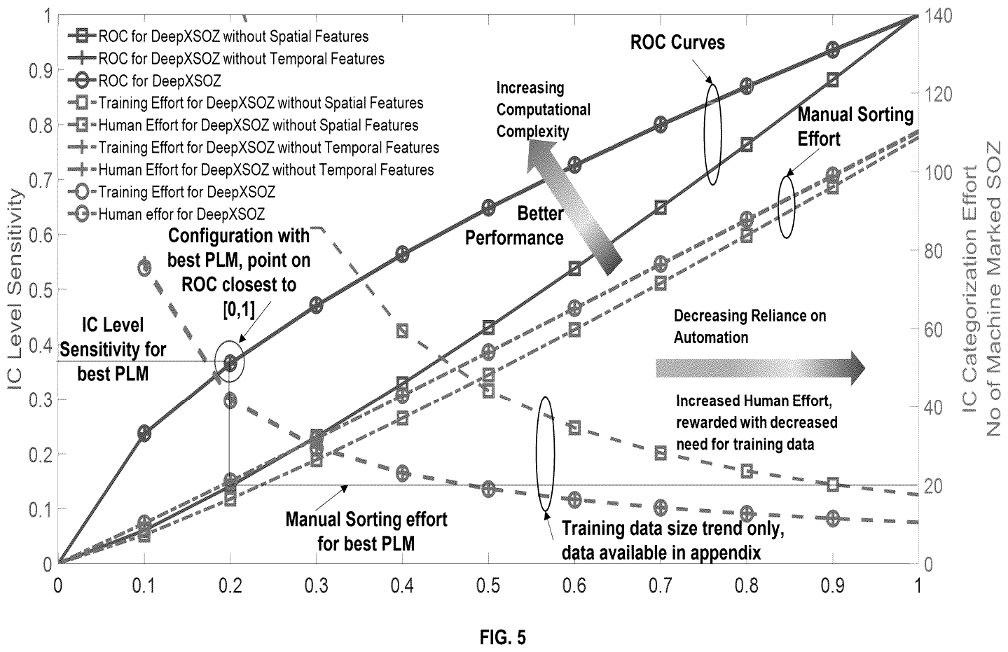

ROC Curve Analysis: DeepXSOZ has the best PLM performance since the indicated point on its ROC curve is closest to the (0,1) point (FIG. 5). The ILM Sensitivity and manual effort is illustrated using the thin vertical and horizontal solid lines. Removing temporal knowledge has little effect on the ROC, however, ROC drastically becomes poorer when the spatial knowledge is removed. As the IC level specificity decreases resulting in more FPS, the effort for manual sorting also increases. This is accompanied by a decrease in the need for training data.

### V. Discussions

1) Efficacy of machine-expert Collaboration for SOZ Identification: The ROC, manual sorting effort and training data requirement curve in FIG. 5 provides a method for personalizing human collaboration with DeepXSOZ according to the expertise of the surgical team. The best performing automated technique (chosen configuration red circle) is DeepXSOZ that automates spatial and temporal knowledge extraction. However, if the surgical team wants to reduce reliance on automation then a configuration with higher IC level sensitivity can be chosen (moving right along the x axis). This will cause an increase in sorting effort to achieve the same PLM, with a reduction in training data requirement. A surgical team with expertise in manual analysis of spatial features can choose the automation strategy in solid line with box markers. To reach similar IC level sensitivity as DeepXSOZ, it will require more manual effort but less training data. The curve also shows that automation of spatial feature analysis has the most improvements on SOZ identification performance.
2) Clinical Significance of Automated Analysis: The rs-fMRI ICA results in approximately 100 ICs. In standard rs-fMRI based pre-surgical screening for PRE children, the entire set of ICs is analyzed by a surgical team to determine which ICs capture blood oxygenation changes due to seizure onset (SOZ localizing IC). The neurosurgeon then determines the location of seizure onset in the brain using the SOZ localizing IC and a recommendation for placement of intracranial EEG leads is made to determine the area to be surgically treated. Given that ICA results in >100 ICs and only <10% are SOZ localizing ICs, manual sorting of rs-fMRI ICs to search for SOZ localizing IC is a significant time commitment by the surgical team resulting in increased cost, reduced availability, and high false positives. An automated whole-brain data-driven SOZ-localizing IC identification technique that is rigorously validated against surgical resection/ablation outcomes, reproducible, equally effective across age, and sex may greatly improve epilepsy care feasibility, morbidity, and mortality. By reducing the number of ICs to be manually sorted and increasing the confidence on the SOZ localizing capability of an IC, DeepXSOZ can potentially alter number and location of SEEG electrodes, and in certain cases possibly need for sEEG itself. Through the automated sorting capability, DeepXSOZ can potentially enable local center expertise in rs-fMRI sorting. This will largely reduce the cost and increase accessibility to pre-surgical screening. Moreover, a >7-fold reduction in manual IC processing need will lead to faster diagnosis and feedback to the surgical team.
3) Sedation in rs-fMRI: Table VII shows that DeepXSOZ patient level sensitivity and IC level SOZ incidence is statistically unaffected by sedation. DeepXSOZs sensitivity is significantly higher, statistically stable, and comparable for both sedated and unsedated patients which further proves the robustness of this technique. Avoiding conscious sedation can be really helpful as it puts additional risks on the children. Although this is an exciting result, however, due to the retrospective nature of the study, the number of un-sedated patients were only 11. This requires a prospective study of design and evaluation of automated techniques in terms of their performance with and without sedation.

TABLE VII
Performance of automated techniques with and without sedation
(N = 11 age matched subjects)

| | | | Sensitivity | SOZ Incidence |
|---|---|---|---|---|
| | Sensitivity | SOZ Incidence | no | no sedation |
| Approach | Sedation | Sedation (SD) | sedation | (SD) |
| DeepXSOZ | 90.9% | 16% (10) | 100% | 16.87% (5.53) |
| LS-SVM | 72.72% | 25.78% (21.5) | 63.63% | 11.74% (10.4) |
| CNN | 45.45% | 7.14% (12.4) | 54.54% | 18.25 (30) |
| EPIK | 63.63% | 5.16% (4.7) | 81.81% | 3.9% (2.89) |

DL techniques on high dimensional data such as biomedical images require a balanced data distribution across the classes of interest. This is often not available for rare diseases where imaging has to be performed with utmost care and safeguards against several potential risks. On the other hand, expert features driven image automation techniques tend to provide high number of FPs.

This disclosure demonstrates the integration of DL with expert knowledge through shallow learning from the specific problem domain which accurately identifies the SOZ in rs-fMRI data for children with PRE. DeepXSOZ achieves significantly higher and consistent results across age, sex, and Engel outcomes in both PLM and ILM from the state-of-the-art solutions and also reduces the number of ICs to be analyzed for pre-surgical evaluation by 7-fold.

### VI. Computer-Implemented System

FIG. 6 is a schematic block diagram of an example computing device 300 that may be used with one or more embodiments described herein, e.g., implementing aspects of the system and methods outlined herein and shown in FIGS. 1-4B.

Computing device 300 comprises one or more network interfaces 310 (e.g., wired, wireless, PLC, etc.), at least one processor 320, and a memory 340 interconnected by a system bus 350, as well as a power supply 360 (e.g., battery, plug-in, etc.). Computing device 300 can also include or otherwise communicate with a display device 330 which can display information including the spatiotemporal imaging data and a list of SOZ ICs outputted by the system 100.

Network interface(s) 310 include the mechanical, electrical, and signaling circuitry for communicating data over the communication links coupled to a communication network. Network interfaces 310 are configured to transmit and/or receive data using a variety of different communication protocols. As illustrated, the box representing network interfaces 310 is shown for simplicity, and it is appreciated that such interfaces may represent different types of network connections such as wireless and wired (physical) connections. Network interfaces 310 are shown separately from power supply 360, however it is appreciated that the interfaces that support PLC protocols may communicate through power supply 360 and/or may be an integral component coupled to power supply 360.

Memory 340 includes a plurality of storage locations that are addressable by processor 320 and network interfaces 310 for storing software programs and data structures associated with the embodiments described herein. In some embodiments, device 300 may have limited memory or no memory (e.g., no memory for storage other than for programs/processes operating on the device and associated caches). Memory 340 can include non-transitory computer readable media, and can include instructions executable by the processor 320 that, when executed by the processor 320, cause the processor 320 to implement aspects of the systems and the methods outlined herein.

Processor 320 comprises hardware elements or logic adapted to execute the software programs (e.g., instructions) and manipulate data structures 345. An operating system 342, portions of which are typically resident in memory 340 and executed by the processor, functionally organizes device 300 by, inter alia, invoking operations in support of software processes and/or services executing on the device. These software processes and/or services may include IC classification processes/services 390, which can include aspects of the methods and/or implementations of various modules described herein, including those outlined in FIGS. 4A and 4B. Note that while IC classification processes/services 390 is illustrated in centralized memory 340, alternative embodiments provide for the process to be operated within the network interfaces 310, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the IC classification processes/services 390 is shown as a standalone process, those skilled in the art will appreciate that this process may be executed as a routine or module within other processes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a processor in communication with a memory, the memory including instructions executable by the processor to:
access spatiotemporal imaging data including independent component data of a plurality of independent components (ICs) of the spatiotemporal imaging data;
determine, for an IC of the plurality of ICs and by a first machine learning model at the processor, a value of a first label of the IC, the first label being indicative of a noise class or a non-noise class associated with the IC;
determine, for the IC and by a second machine learning model at the processor, a value of a second label of the IC based on a plurality of features of the IC, the second label being indicative of a Resting State Network (RSN) activity class or a Seizure Onset Zone (SOZ) activity class associated with the IC; and
assign an output label of the IC based on the value of the first label and the value of the second label associated with the IC, the output label indicating the noise class, the RSN activity class, or the SOZ activity class.

2. The system of claim 1, the memory further including instructions executable by the processor to:
generate a list of ICs having the output label indicating the SOZ activity class.

3. The system of claim 1, the memory further including instructions executable by the processor to:
assign a first output value indicating the noise class to the output label based on the value of the first label associated with the IC, the value of the first label associated with the IC indicating the noise class.

4. The system of claim 1, the memory further including instructions executable by the processor to:
assign a second output value indicating the SOZ activity class to the output label based on the value of the second label associated with the IC, the value of the second label associated with the IC indicating the SOZ activity class.

5. The system of claim 4, the value of the first label associated with the IC indicating the non-noise class.

6. The system of claim 4, the memory further including instructions executable by the processor to:
compare, for the IC, a posterior probability associated with the second label with respect to a threshold value, the first label of the IC indicating the noise class and the second label of the IC indicating the SOZ activity class; and
assign, based on comparison between the posterior probability and the threshold value, the second output value indicating the SOZ activity class to the output label based on the value of the second label associated with the IC, the posterior probability exceeding the threshold value.

7. The system of claim 1, the memory further including instructions executable by the processor to:
assign a third output value indicating the RSN activity class to the output label based on the value of the second label associated with the IC, the value of the second label associated with the IC indicating the RSN activity class and the value of the first label indicating the non-noise class.

8. The system of claim 1, the first machine learning model including a vision-based deep learning neural network model operable to evaluate noisiness of the IC based on the independent component data associated with the IC.

9. The system of claim 1, the memory further including instructions executable by the processor to:
extract, prior to determining the value of the second label, the plurality of features of the IC from the independent component data associated with the IC, including:
information representing clusters present within the spatiotemporal imaging data and associated with the IC;
information representing clusters present within the spatiotemporal imaging data associated with the IC that overlap within white matter areas and/or gray matter areas;
information representing sparsity of an activelet basis representation of the IC; and
information representing sparsity of a sine representation of the IC.

10. The system of claim 1, the second machine learning model being a linear-support vector machine model operable to evaluate an activity state of the IC based on the plurality of features associated with the IC.

11. The system of claim 1, the second machine learning model having been trained to determine the value of the second label based on features of the IC based on a training dataset, the training dataset including training IC data representing a plurality of training ICs having a plurality of training features, a first subset of the plurality of training ICs being associated with the RSN activity class and a second subset of the plurality of training ICs being associated with the SOZ activity class.

12. A method, comprising:
accessing, by a processor in communication with a memory, spatiotemporal imaging data including independent component data of a plurality of independent components (ICs) of the spatiotemporal imaging data;
determining, for an IC of the plurality of ICs and by a first machine learning model at the processor, a value of a first label of the IC, the first label being indicative of a noise class or a non-noise class associated with the IC;
determining, for the IC and by a second machine learning model at the processor, a value of a second label of the IC based on a plurality of features of the IC, the second label being indicative of a Resting State Network (RSN) activity class or a Seizure Onset Zone (SOZ) activity class associated with the IC; and
assigning an output label of the IC based on the value of the first label and the value of the second label associated with the IC, the output label indicating the noise class, the RSN activity class, or the SOZ activity class.

13. The method of claim 12, further comprising:
generating a list of ICs having the output label indicating the SOZ activity class.

14. The method of claim 12, further comprising:
assigning a first output value indicating the noise class to the output label based on the value of the first label associated with the IC, the value of the first label associated with the IC indicating the noise class.

15. The method of claim 12, further comprising:
assigning a second output value indicating the SOZ activity class to the output label based on the value of the second label associated with the IC, the value of the second label associated with the IC indicating the SOZ activity class.

16. The method of claim 15, the value of the first label associated with the IC indicating the non-noise class.

17. The method of claim 12, further comprising:
comparing, for the IC, a posterior probability associated with the second label with respect to a threshold value, the first label of the IC indicating the noise class and the second label of the IC indicating the SOZ activity class; and
assigning, based on comparison between the posterior probability and the threshold value, the second output value indicating the SOZ activity class to the output label based on the value of the second label associated with the IC, the posterior probability exceeding the threshold value.

18. The method of claim 12, further comprising:
assigning a third output value indicating the RSN activity class to the output label based on the value of the second label associated with the IC, the value of the second label associated with the IC indicating the RSN activity class and the value of the first label indicating the non-noise class.

19. The method of claim 12, further comprising:
extracting, prior to determining the value of the second label, the plurality of features of the IC from the independent component data associated with the IC, including:
information representing clusters present within the spatiotemporal imaging data and associated with the IC;
information representing clusters present within the spatiotemporal imaging data associated with the IC that overlap within white matter areas and/or gray matter areas;
information representing sparsity of an activelet basis representation of the IC; and
information representing sparsity of a sine representation of the IC.

20. The method of claim 12, where the second machine learning model being a linear-support vector machine model operable to evaluate an activity state of the IC based on the plurality of features associated with the IC.
